(19) United States
(12) Patent Application Publication
(10) Pub. No.: US 2015/0177311 A1
(43) Pub. Date: Jun. 25, 2015

(54) Methods and Systems for Evaluating IGZO with Respect to NBIS

(71) Applicant: Intermolecular, Inc., San Jose, CA (US)

(72) Inventor: Khaled Ahmed, Anaheim, CA (US)

(73) Assignee: Intermolecular, Inc., San Jose, CA (US)

(21) Appl. No.: 14/135,408

(22) Filed: Dec. 19, 2013

(51) Int. Cl. G01R 31/26; G01N 27/00; G01N 33/00; H01L 29/26

(52) U.S. Cl. CPC G01R 31/2648; H01L 29/263; G01N 27/00; G01N 33/00; G01N 2033/0095

(57) ABSTRACT

Embodiments described herein provide methods and systems for evaluating indium-gallium-zinc oxide (IGZO) with respect to negative bias illumination stress (NBIS). A plurality of IGZO devices is formed. Each of the plurality of IGZO devices includes a semiconductor substrate and an IGZO layer formed above the semiconductor substrate. A processing condition used to form at least two of the plurality of IGZO devices is varied in a combinatorial manner. A bias is applied to the semiconductor substrate of each of the plurality of IGZO devices. A current flow through each of the plurality of IGZO devices while the bias is applied is measured.

### TECHNICAL FIELD

The present invention relates to indium-gallium-zinc oxide (IGZO) devices. More particularly, this invention relates to methods and systems for evaluating IGZO with respect to negative bias illumination stress (NBIS).

### BACKGROUND OF THE INVENTION

Indium-gallium-zinc oxide (IGZO) devices, such as IGZO thin-film transistors (TFTs) have attracted a considerable amount of attention due to the associated low cost, room temperature manufacturing processes with good uniformity control, high mobility for high speed operation, and the compatibility with transparent, flexible, and light display applications. Due to these attributes, IGZO TFTs may even be favored over low cost amorphous silicon TFTs and relatively high mobility polycrystalline silicon TFT for display device applications. IGZO devices typically utilize amorphous IGZO (a-IGZO).

Recent developments in the field suggest that the use of crystalline IGZO may provide improved electrical and chemical stability in certain conditions. However, IGZO TFTs may lack electrical stability under negative bias illumination stress (NBIS). A complete understanding of why this instability occurs in oxide semiconductors is crucial to prevent it from happening, and there have been many attempts to explain its origin, the major two arguments being the charge trapping model and the ion diffusion model. Recent reports support the charge trapping model, where holes generated in the IGZO layer upon illumination tunnel into traps in the gate dielectric when the gate electrode is negatively biased and cause a negative threshold shift.

Using conventional methods, assessing NBIS in IGZO TFTs typically requires about one hour of measurement time per transistor. Such methods are not suitable, particularly when attempting to test multiple devices/materials for performance with respect to NBIS.

### BRIEF DESCRIPTION OF THE DRAWINGS

To facilitate understanding, identical reference numerals have been used, where possible, to designate identical elements that are common to the figures. The drawings are not to scale and the relative dimensions of various elements in the drawings are depicted schematically and not necessarily to scale.

The techniques of the present invention can readily be understood by considering the following detailed description in conjunction with the accompanying drawings, in which:

FIG. 1 is a schematic diagram for implementing combinatorial processing and evaluation.

FIG. 2 is a simplified schematic diagram illustrating a general methodology for combinatorial process sequence integration.

FIG. 3 is a cross-sectional view of a substrate according to some embodiments.

FIG. 4 is a cross-sectional view of the substrate of FIG. 1 with a dielectric layer formed above.

FIG. 5 is a cross-sectional view of the substrate of FIG. 4 with a test layer formed above the dielectric layer.

FIG. 6 is a cross-sectional view of the substrate of FIG. 5 with an array of conductors formed above the test layer.

FIG. 7 is a cross-sectional schematic view of a system for evaluating indium-gallium-zinc oxide (IGZO) with respect to negative bias illumination stress (NBIS).

FIG. 8 is a cross-sectional view of an IGZO device according to some embodiments.

FIG. 9 is a simplified cross-sectional diagram of a physical vapor deposition (PVD) tool according to some embodiments.

FIG. 10 is a flow chart illustrating a method for evaluating IGZO with respect to NBIS according to some embodiments.

### DETAILED DESCRIPTION

A detailed description of one or more embodiments is provided below along with accompanying figures. The detailed description is provided in connection with such embodiments, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For the purpose of clarity, technical material that is known in the technical fields related to the embodiments has not been described in detail to avoid unnecessarily obscuring the description.

The term “horizontal” as used herein will be understood to be defined as a plane parallel to the plane or surface of the substrate, regardless of the orientation of the substrate. The term “vertical” will refer to a direction perpendicular to the horizontal as previously defined. Terms such as “above”, “below”, “bottom”, “top”, “side” (e.g. sidewall), “higher”, “lower”, “upper”, “over”, and “under”, are defined with respect to the horizontal plane. The term “on” means there is direct contact between the elements. The term “above” will allow for intervening elements.

The manufacture of various devices, such as indium-gallium-zinc oxide (IGZO) devices (e.g., IGZO thin-film transistors (TFTs), entails the integration and sequencing of many unit processing steps. For example, device manufacturing typically includes a series of processing steps such as cleaning, surface preparation, deposition, patterning, etching, thermal annealing, and other related unit processing steps. The precise sequencing and integration of the unit processing steps enables the formation of functional devices meeting desired performance metrics such as efficiency, power production, and reliability.

As part of the discovery, optimization and qualification of each unit process, it is desirable to be able to i) test different materials, ii) test different processing conditions within each unit process module, iii) test different sequencing and integration of processing modules within an integrated processing tool, iv) test different sequencing of processing tools in executing different process sequence integration flows, and combinations thereof in the manufacture of devices such as integrated circuits. In particular, there is a need to be able to test i) more than one material, ii) more than one processing condition, iii) more than one sequence of processing conditions, iv) more than one process sequence integration flow, and combinations thereof, collectively known as “combinatorial process sequence integration,” on a single monolithic substrate (e.g., an integrated or short-looped wafer) without the need of consuming the equivalent number of monolithic substrates per material(s), processing condition(s), sequence(s) of processing conditions, sequence(s) of processes, and combinations thereof. This can greatly improve both the speed and reduce the costs associated with the discovery, implementation, optimization, and qualification of material(s), process(es), and process integration sequence(s) required for manufacturing.

Systems and methods for High Productivity Combinatorial (HPC) processing are described in U.S. Pat. No. 7,544,574, filed on Feb. 10, 2006, U.S. Pat. No. 7,824,935, filed on Jul. 2, 2008, U.S. Pat. No. 7,871,928, filed on May 4, 2009, U.S. Pat. No. 7,902,063, filed on Feb. 10, 2006, and U.S. Pat. No. 7,947,531, filed on Aug. 28, 2009, which are all herein incorporated by reference. Systems and methods for HPC processing are further described in U.S. patent application Ser. No. 11/352,077, filed on Feb. 10, 2006, claiming priority from Oct. 15, 2005, U.S. patent application Ser. No. 11/419,174, filed on May 18, 2006, claiming priority from Oct. 15, 2005, U.S. patent application Ser. No. 11/674,132, filed on Feb. 12, 2007, claiming priority from Oct. 15, 2005, and U.S. patent application Ser. No. 11/674,137, filed on Feb. 12, 2007, claiming priority from Oct. 15, 2005 which are all herein incorporated by reference.

HPC processing techniques have been successfully adapted to wet chemical processing such as etching and cleaning. HPC processing techniques have also been successfully adapted to deposition processes such as physical vapor deposition (PVD), atomic layer deposition (ALD), and chemical vapor deposition (CVD).

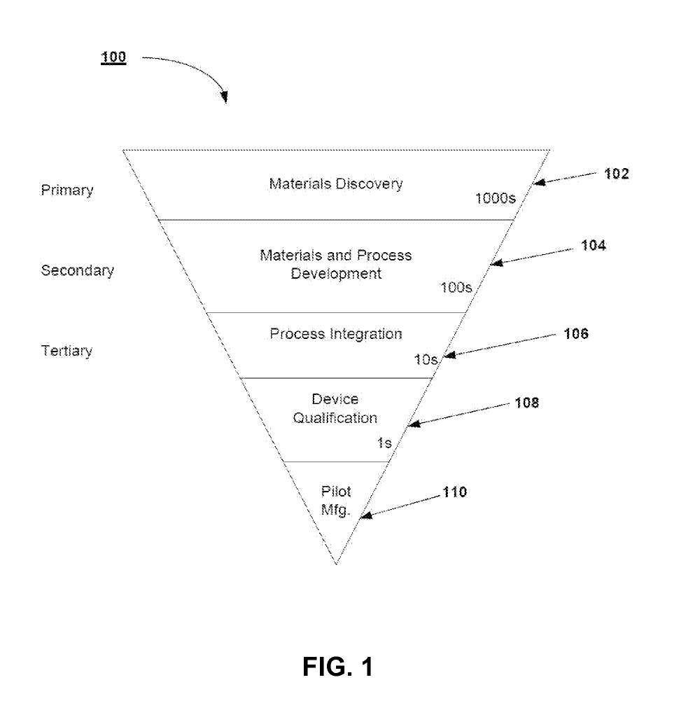

FIG. 1 illustrates a schematic diagram 100 for implementing combinatorial processing and evaluation using primary, secondary, and tertiary screening. The schematic diagram 100 illustrates that the relative number of combinatorial processes run with a group of substrates decreases as certain materials and/or processes are selected. Generally, combinatorial processing includes performing a large number of processes during a primary screen, selecting promising candidates from those processes, performing the selected processing during a secondary screen, selecting promising candidates from the secondary screen for a tertiary screen, and so on. In addition, feedback from later stages to earlier stages can be used to refine the success criteria and provide better screening results.

For example, thousands of materials are evaluated during a materials discovery stage 102. Materials discovery stage 102 is also known as a primary screening stage performed using primary screening techniques. Primary screening techniques may include dividing substrates into coupons and depositing materials using varied processes. The materials are then evaluated, and promising candidates are advanced to the secondary screen, or materials and process development stage 104. Evaluation of the materials is performed using metrology tools such as electronic testers and imaging tools (i.e., microscopes).

The materials and process development stage 104 may evaluate hundreds of materials (i.e., a magnitude smaller than the primary stage) and may focus on the processes used to deposit or develop those materials. Promising materials and processes are again selected, and advanced to the tertiary screen or process integration stage 106, where tens of materials and/or processes and combinations are evaluated. The tertiary screen or process integration stage 106 may focus on integrating the selected processes and materials with other processes and materials.

The most promising materials and processes from the tertiary screen are advanced to device qualification 108. In device qualification, the materials and processes selected are evaluated for high volume manufacturing, which normally is conducted on full substrates within production tools, but need not be conducted in such a manner. The results are evaluated to determine the efficacy of the selected materials and processes. If successful, the use of the screened materials and processes can proceed to pilot manufacturing 110.

The schematic diagram 100 is an example of various techniques that may be used to evaluate and select materials and processes for the development of new materials and processes. The descriptions of primary, secondary, etc. screening and the various stages 102-110 are arbitrary and the stages may overlap, occur out of sequence, be described and be performed in many other ways.

This application benefits from High Productivity Combinatorial (HPC) techniques described in U.S. patent application Ser. No. 11/674,137m filed on Feb. 12, 2007, which is hereby incorporated for reference in its entirety. Portions of the '137 application have been reproduced below to enhance the understanding of the present invention. The embodiments described herein enable the application of combinatorial techniques to process sequence integration in order to arrive at a globally optimal sequence of, for example, device manufacturing operations by considering interaction effects between the unit manufacturing operations, the process conditions used to effect such unit manufacturing operations, hardware details used during the processing, as well as materials characteristics of components utilized within the unit manufacturing operations. Rather than only considering a series of local optimums, i.e., where the best conditions and materials for each manufacturing unit operation is considered in isolation, the embodiments described below consider interactions effects introduced due to the multitude of processing operations that are performed and the order in which such multitude of processing operations are performed when fabricating a device. A global optimum sequence order is therefore derived and as part of this derivation, the unit processes, unit process parameters and materials used in the unit process operations of the optimum sequence order are also considered.

The embodiments described further analyze a portion or sub-set of the overall process sequence used to manufacture a device. Once the subset of the process sequence is identified for analysis, combinatorial process sequence integration testing is performed to optimize the materials, unit processes, hardware details, and process sequence used to build that portion of the device or structure. During the processing of some embodiments described herein, structures are formed on the processed substrate(s) that are equivalent to the structures formed during actual production of the device. For example, such structures may include, but would not be limited to, contact layers, buffer layers, absorber layers, or any other series of layers or unit processes that create an intermediate structure found on devices. While the combinatorial processing varies certain materials, unit processes, hardware details, or process sequences, the composition or thickness of the layers or structures or the action of the unit process, such as cleaning, surface preparation, deposition, surface treatment, etc. is substantially uniform through each discrete region. Furthermore, while different materials or unit processes may be used for corresponding layers or steps in the formation of a structure in different regions of the substrate(s) during the combinatorial processing, the application of each layer or use of a given unit process is substantially consistent or uniform throughout the different regions in which it is intentionally applied. Thus, the processing is uniform within a region (inter-region uniformity) and between regions (intra-region uniformity), as desired. It should be noted that the process can be varied between regions, for example, where a thickness of a layer is varied or a material may be varied between the regions, etc., as desired by the design of the experiment.

The result is a series of regions on the substrate, or substrates, that contain structures or unit process sequences that have been uniformly applied within that region and, as applicable, across different regions. This process uniformity allows comparison of the properties within and across the different regions such that the variations in test results are due to the varied parameter (e.g., materials, unit processes, unit process parameters, hardware details, or process sequences) and not the lack of process uniformity. In the embodiments described herein, the positions of the discrete regions on the substrate(s) can be defined as needed, but are preferably systematized for ease of tooling and design of experimentation. In addition, the number, variants and location of structures within each region are designed to enable valid statistical analysis of the test results within each region and across regions to be performed.

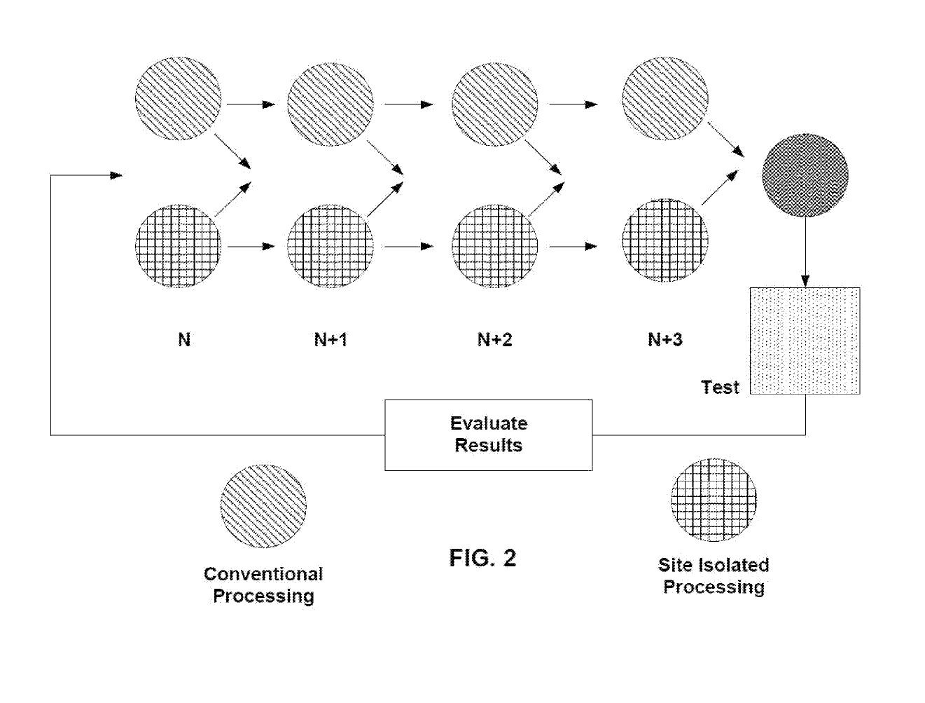

FIG. 2 is a simplified schematic diagram illustrating a general methodology for combinatorial process sequence integration that includes site isolated processing and/or conventional processing in accordance with one embodiment of the invention. In one embodiment, the substrate(s) is initially processed using conventional process N. In one exemplary embodiment, the substrate is then processed using site isolated process N+1. During site isolated processing, an HPC module may be used, such as the HPC module described in U.S. patent application Ser. No. 11/352,077, filed on Feb. 10, 2006. The substrate(s) can then be processed using site isolated process N+2, and thereafter processed using conventional process N+3. Testing is performed and the results are evaluated. The testing can include physical, chemical, acoustic, magnetic, electrical, optical, etc. tests. From this evaluation, a particular process from the various site isolated processes (e.g. from steps N+1 and N+2) may be selected and fixed so that additional combinatorial process sequence integration may be performed using site isolated processing for either process N or N+3. For example, a next process sequence can include processing the substrate using site isolated process N, conventional processing for processes N+1, N+2, and N+3, with testing performed thereafter.

It should be appreciated that various other combinations of conventional and combinatorial processes can be included in the processing sequence with regard to FIG. 2. That is, the combinatorial process sequence integration can be applied to any desired segments and/or portions of an overall process flow. Characterization, including physical, chemical, acoustic, magnetic, electrical, optical, etc. testing, can be performed after each process operation, and/or series of process operations within the process flow as desired. The feedback provided by the testing is used to select certain materials, processes, process conditions, and process sequences and eliminate others. Furthermore, the above flows can be applied to entire monolithic substrates, or portions of monolithic substrates such as coupons.

Under combinatorial processing operations the processing conditions at different regions can be controlled independently. Consequently, process material amounts, reactant species, processing temperatures, processing times, processing pressures, processing flow rates, processing powers, processing reagent compositions, the rates at which the reactions are quenched, deposition order of process materials, process sequence steps, hardware details, etc., can be varied from region to region on the substrate, or from substrate to substrate. Thus, for example, when exploring materials, a processing material delivered to a first and second region can be the same or different. If the processing material delivered to the first region is the same as the processing material delivered to the second region, this processing material can be offered to the first and second regions on the substrate at different concentrations. In addition, the material can be deposited under different processing parameters. Parameters which can be varied include, but are not limited to, process material amounts, reactant species, processing temperatures, processing times, processing pressures, processing flow rates, processing powers, processing reagent compositions, the rates at which the reactions are quenched, atmospheres in which the processes are conducted, an order in which materials are deposited, hardware details of the gas distribution assembly, etc. It should be appreciated that these process parameters are exemplary and not meant to be an exhaustive list as other process parameters commonly used in device manufacturing may be varied.

Embodiments described herein provide methods and systems for evaluating IGZO with respect to negative bias illumination stress (NBIS) in a rapid and efficient manner. In some embodiments, multiple test devices (or IGZO devices) are formed, each of which includes IGZO. In some embodiments, the test devices each include a semiconductor substrate (e.g., P++ doped silicon), a dielectric layer (e.g., silicon oxide or silicon nitride) above the substrate, an IGZO layer above the dielectric layer, and one or more conductors above the IGZO layer. In some embodiments, the test devices (or at least the IGZO layers therein) are formed in a combinatorial manner. That is, in some embodiments, each test device is formed using a respective set of processing conditions.

While a bias is applied to the substrate and the devices are illuminated, a current flow through the devices is measured. The amount of current flow through the devices may be indicative of the performance of the devices (or the IGZO therein) with respect to NBIS. The test devices (or IGZO) with relatively low current flow (and/or the processing conditions used to form the test devices/IGZO) may be selected for further testing and/or use in (and/or to form) IGZO devices, such as TFTs.

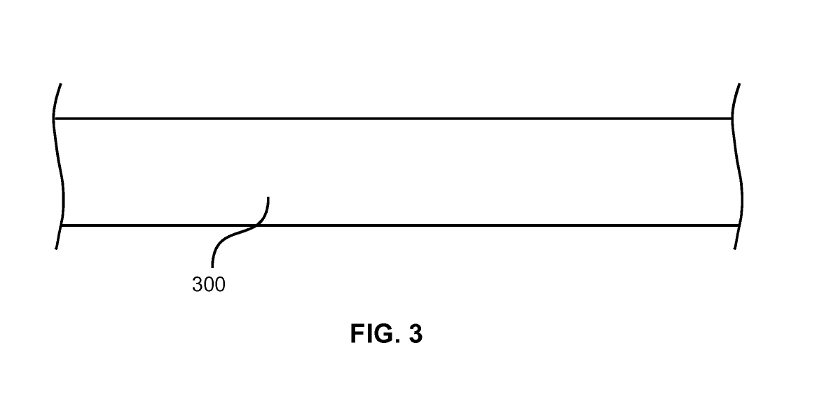

FIGS. 3-6 illustrate a method for forming an IZGO test device (or simply an IGZO device) according to some embodiments. Referring to FIG. 3, a substrate 300 is provided. The substrate 300 may be made of a semiconductor material. In some embodiments, the substrate 300 includes (or is made of) silicon, and may include a relatively high concentration (e.g., 1×10¹⁹ cm⁻³ or greater) of a P-type dopant, such as boron (e.g., P++ doped silicon). The substrate 300 may have a thickness of, for example, between about 0.01 centimeters (cm) and about 0.5 cm. Although only a portion of the substrate 300 is shown, it should be understood that the substrate 100 may have a width of, for example, between about 5.0 cm and about 4.0 meters (m).

It should be understood that the various components above the substrate 300, such those described below, may be formed using processing techniques suitable for the particular materials being deposited, such as physical vapor deposition (PVD) (e.g., co-sputtering in some embodiments), chemical vapor deposition (CVD), plasma-enhanced (PECVD), electroplating, etc.

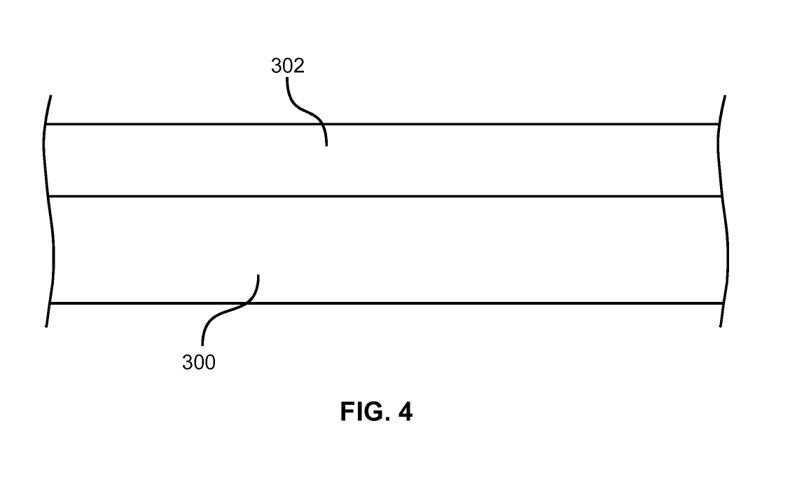

Referring to FIG. 4, a dielectric layer (or a gate dielectric layer) 302 is formed above the substrate 300. In some embodiments, the dielectric layer 302 includes (or is made of) silicon nitride, silicon oxide, or a combination thereof. The dielectric layer 302 may have a thickness of, for example, between about 200 nanometers (nm) and about 400 nm, such as about 300 nanometers. In some embodiments, the dielectric layer 302 is formed using PVD.

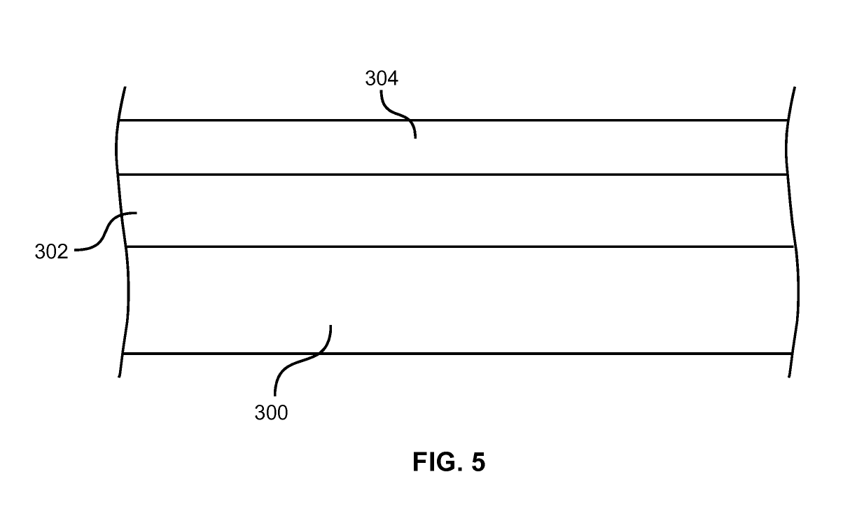

As shown in FIG. 5, a test layer 304 is then formed above the dielectric layer 302. In some embodiments, the test layer includes (or is made of) IGZO. The IGZO may a ratio of the respective elements of, for example, 1:1:1:1-3. In some embodiments, the IGZO within the test layer 304 is deposited as amorphous IGZO (a-IGZO). However, in some embodiments, the IGZO is formed in such a way to enhance the crystal structure thereof. In some embodiments, the test layer 304 is formed using PVD. The IGZO may be deposited from a single target that includes indium, gallium, and zinc (e.g., an indium-gallium-zinc alloy target or an IGZO target), but two or more targets may also used (e.g., co-sputtering with an indium-zinc target and a gallium target). The test layer 304 may have a thickness of, for example, between about 30 nm and about 100 nm, such as about 50 nm.

In some embodiments, the test layer 304 (as well as the substrate 300 and the dielectric layer 302) may undergo an annealing process. In some embodiments, the annealing process includes a relatively low temperature (e.g., less than about 600° C., preferably less than about 450° C.) heating process in, for example, an ambient gaseous environment (e.g., nitrogen, oxygen, or ambient/air) to, for example, (further) enhance the crystalline structure of the IGZO. The heating process may occur for between about 1 minute and about 200 minutes. After the annealing (or heating) process, the IGZO may (substantially) include crystalline IGZO (c-IGZO). As used herein a “crystalline” material (e.g., c-IGZO) may be considered to be one that is more than 30% crystalline by volume, as determined by a technique such as X-ray Diffraction (XRD). In some embodiments, the c-IGZO is c-axis aligned crystal (CAAC) IGZO, as is commonly understood.

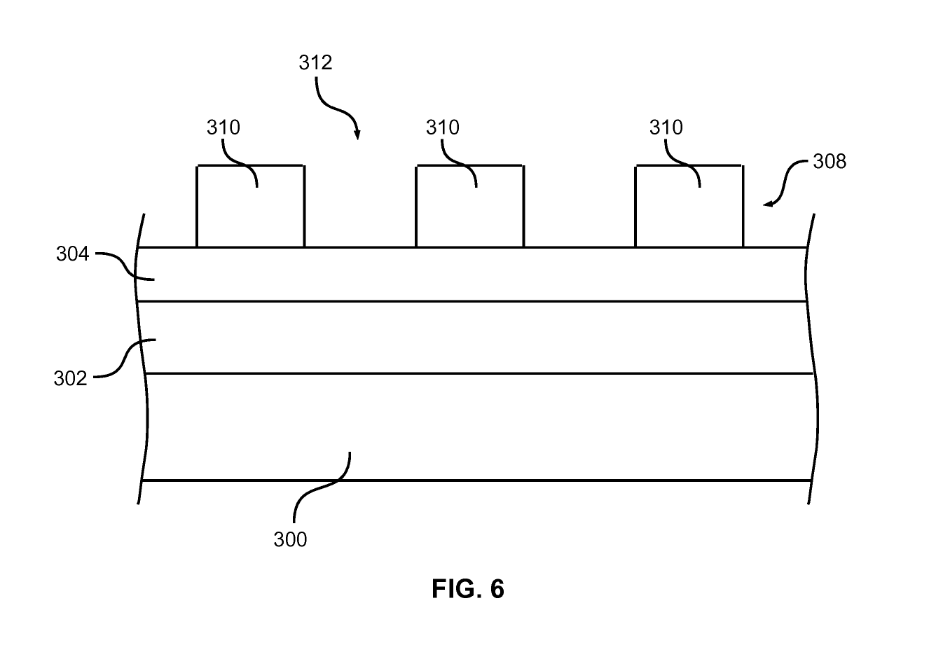

Referring now to FIG. 6, a conductor array 308 is then formed above the test layer 304. As shown, the conductor array 308 includes multiple individual conductors 310. In some embodiments, the conductors 310 include (or are made of) a transparent conductive material, such as transparent graphene, indium-tin oxide (ITO), zinc oxide. In some embodiments, the conductors include platinum. The conductors 310 may have a thickness of, for example, between about 400 nm and about 0.5 cm. In some embodiments, the conductors 310 are formed using PVD in combination with a shadow mask, as is commonly understood. Although only shown in cross-section, each of the conductors 310 may be substantially square in shape (i.e., when viewed from above) with side lengths of, for example, between about 400 nm and about 0.5 cm. Additionally, the array 308 may include multiple rows of conductors 308 formed above the test layer 304.

The formation of the conductor array 308 may substantially complete the formation of a test device (or an IGZO device) 312 according to some embodiments. In some embodiments, multiple test devices 312 are formed in combinatorial manner (e.g., by creating differences/variations in the processing conditions used during the formation process), particularly with respect to the IGZO within the test layer 304, in accordance with the various characteristics of combinatorial processing described above. As described below, the test devices 312 may be used to, for example, evaluate the performance of the IGZO within the test layer 304 with respect to NBIS.

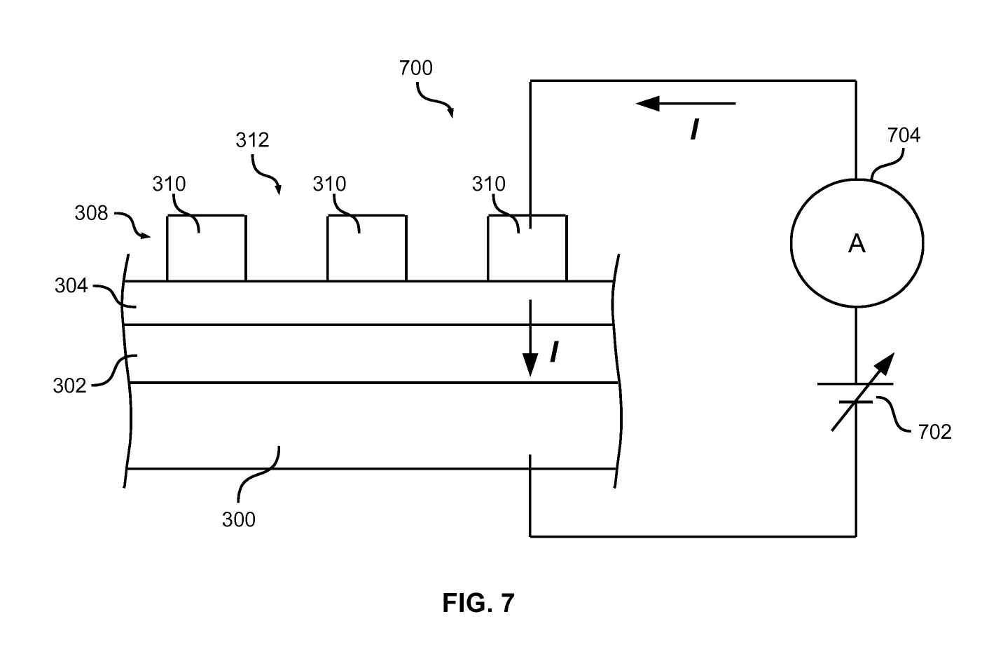

FIG. 7 schematically illustrates a system 700 for evaluating the performance of test devices 312 (or more particularly, the IGZO therein) with respect to NBIS in according to some embodiments. As shown, the system 700 includes a voltage supply 702, an ammeter 704, and (one of) the test device(s) 312. As shown, the voltage supply 702 and the ammeter 704 are electrically connected in series, and in the depicted embodiment, a terminal of the voltage supply 702 is electrically connected to the substrate 300 of the test device 312 and a terminal of the ammeter 704 is electrically connected to one of the conductors 310 (or the array 308 as a whole).

In some embodiments, to evaluate the IGZO with respect to NBIS, the test device 312 is first exposed to a first amount of illumination. The first amount of illumination may include positioning the test device 312 in a low light (or dark) environment such that substantially no light is directed onto (or shone) onto the test device 312. While being exposed to the first amount of illumination, a bias (or voltage) is applied to the substrate 300. In some embodiments, the bias is between about −10 V and about −20 V.

The ammeter 704 is then used to measure the current (e.g., direct current (DC)) flow through the test device 312. More particularly, the ammeter 704 is used to measure the DC current flow through the substrate 300, the dielectric layer 302, the test layer 304, and the conductor(s) 310 (i.e., when the test device 312 is exposed to the first amount of illumination and the bias is applied). The barrier height for valence band holes at the interface between the dielectric layer 302 and the test layer 304 is zero. Electron-hole pairs (ehp) generated in the test layer 304 upon illumination result in a current flow in the direction indicated in FIG. 7. The measurement of this current may be performed for a relatively brief amount of time (e.g., less than 10 seconds). Additionally, this current (i.e., I_{dark}) may be considered to correspond to a “baseline” current flowing through the test device 312 without any NBIS (i.e., because little or no illumination is provided).

The test device 312 is then exposed to a second amount of illumination (e.g., greater than the first amount of illumination). The second amount of illumination may be provided by directing light (e.g., monochromatic light) onto the test device. In some embodiments, the light has a wavelength of, for example, between about 400 nm and about 600 nm. While being exposed to the second amount of illumination, a bias (or voltage) is applied to the substrate 300. In some embodiments, the bias is between about −10 V and about −20 V.

The ammeter 704 is then used to measure the current (e.g., direct current (DC)) flow through the test device 312 (i.e., while being exposed to the second amount of illumination, with the bias being applied to the substrate 300). More particularly, the ammeter 704 is used to measure the DC current flow through the substrate 300, the dielectric layer 302, the test layer 304, and the conductor(s) 310. The measurement of this current may be performed for a relatively brief amount of time (e.g., less than 10 seconds). Additionally, this current (i.e., I_{light}) may be considered to correspond to a “NBIS” current flowing through the test device 312 (i.e., because a substantial amount of illumination is provided). As an example, I_{light} may be about 100 picoampere (pA). However, this value may vary depending on, for example, the dimensions and other characteristics of the test devices 312 (e.g., width, thickness of the layers/components, etc.)

In some embodiments, I_{light} is then compared to I_{dark} to make sure that the magnitude of I_{light} is greater (e.g., about 10× or more) the magnitude of I_{dark} (e.g., to ensure that the light is generating electron-hole pairs and the measured current is due to “hole transport or tunneling”).

If the magnitude of I_{tight} is greater than (e.g., at least 10×) the magnitude of I_{dark}, I_{light} may be used as a “proxy” for the density of electron-hole pairs generated by the (second amount) of illumination, as electron holes will flow into the dielectric layer 302 and get trapped. These trapped electron holes cause a threshold voltage shift when the IGZO is used in a TFT under normal operating conditions. The magnitude of the current represents the density of holes generated in the test layer 304. “Poor quality” IGZO layers will have higher density of ehp generation, and higher current flow under a negative bias condition. “Good quality” IGZO layers will have lower density of ehp generation, and lower current flow under a negative bias condition.

Using the method described above, all of the test devices 312 formed (e.g., using combinatorial processing) may be tested, and those with the lowest measured current (i.e., I_{light}), or more particularly, the IGZO used therein (and/or the processing conditions used to form those test devices/IGZO), may be selected for further testing and/or use in (and/or to form) IGZO devices (e.g., TFTs), such as the example described below.

In some embodiments, the test devices 312 (and/or the IGZO used therein and/or the processing conditions use to form the devices/IGZO) that are selected are those with a measured current (i.e., I_{light}) below a threshold. The threshold may a particular value (e.g., 100 pA, or more or less) depending on the particular application for which the devices/IGZO are being tested. However, it should be understood that the threshold may simply be a pseudo-arbitrary, user-defined threshold used to eliminate the test devices/IGZO which are least likely to be suitable for the intended application(s) (e.g., those with relatively high I_{light} measurements).

Using this method, the performance of multiple devices (and/or IGZO layers) with respect to NBIS may be quickly and efficiently evaluated (or at least compared with to each other).

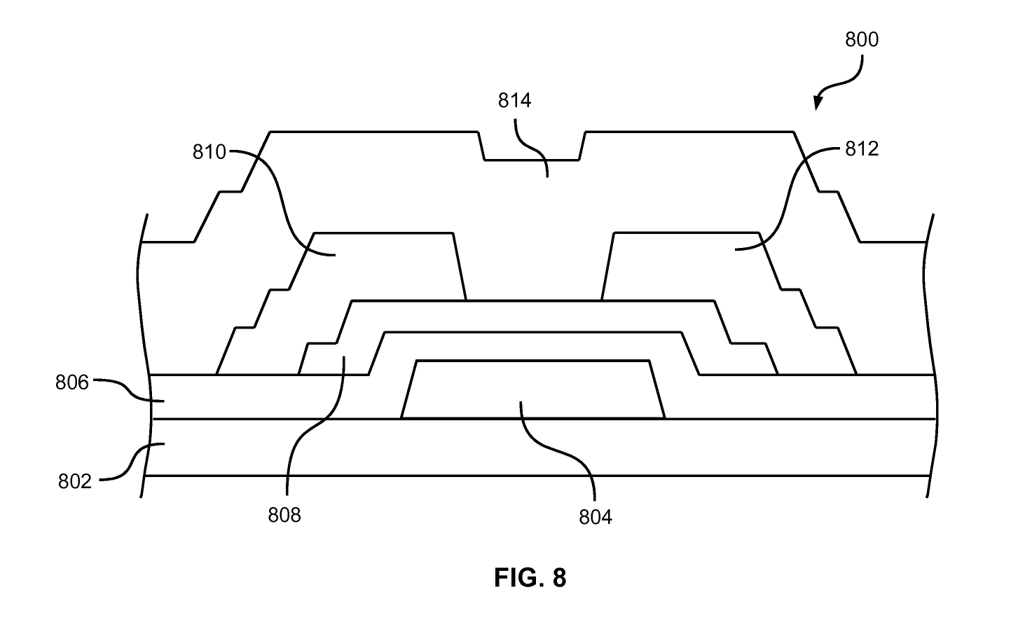

FIG. 8 illustrates an IGZO device (e.g., an IGZO TFT) 800 according to some embodiments. The IGZO device 800 is merely intended as an example in which the evaluated IGZO described may be utilized. The IGZO device 800 includes a substrate 802, a gate electrode 804, a gate dielectric layer 806, an IGZO channel layer 808, a source electrode 810, a drain electrode 812, and a passivation layer 814.

In some embodiments, the substrate 802 is transparent and is made of, for example, glass. The substrate 802 may have a thickness of, for example, between about 0.01 centimeters (cm) and about 0.5 cm. Although only a portion of the substrate 100 is shown, it should be understood that the substrate 100 may have a width of, for example, between about 5.0 cm and about 4.0 meters (m). Although not shown, in some embodiments, the substrate 100 may have a dielectric layer (e.g., silicon oxide) formed above an upper surface thereof. In such embodiments, the components described below are formed above the dielectric layer. Also, in some embodiments, the substrate 100 is at least partially made of a of a semiconductor material (e.g., silicon, germanium, gallium arsenide, etc.). For example, in some embodiments, the substrate includes glass with a layer of semiconductor material formed thereon.

The gate electrode 804 is formed above the substrate 802. In some embodiments, the gate electrode 804 is made of a conductive material, such as copper, silver, aluminum, manganese, molybdenum, or a combination thereof. The gate electrode 804 may have a thickness of, for example, between about 20 nm and about 500 nm.

It should be understood that the various components above the substrate 100, such as the gate electrode 102 and those described below, are formed using processing techniques suitable for the particular materials being deposited, such as physical vapor deposition (PVD) (e.g., co-sputtering in some embodiments), chemical vapor deposition (CVD), plasma-enhanced (PECVD), electroplating, etc. Furthermore, it should be understood that the various components formed above the substrate 802, such as the gate electrode 804, may be sized and shaped using a photolithography process and an etching process, as is commonly understood, such that the components are formed above selected regions of the substrate 802.

Referring to FIG. 8, the gate dielectric layer 806 is formed above the gate electrode 804 and the exposed portions of the substrate 802. The gate dielectric layer 806 may be made of, for example, silicon oxide, silicon nitride, or a high-k dielectric (e.g., having a dielectric constant greater than 3.9), such as zirconium oxide, hafnium oxide, or aluminum oxide. In some embodiments, the gate dielectric layer 806 has a thickness of, for example, between about 10 nm and about 500 nm.

The IGZO channel layer 808 is formed above the gate dielectric layer 806. The IGZO channel layer 808 may be made of IGZO in which a ratio of the respective elements is, for example, 1:1:1:1-3. In some embodiments, the IGZO within the IGZO channel layer 808 is deposited as amorphous IGZO (a-IGZO). In some embodiments, the IGZO channel layer 808 is formed using PVD. The IGZO may be deposited from a single target that includes indium, gallium, and zinc (e.g., an indium-gallium-zinc alloy target or an IGZO target), but two or more targets may also used (e.g., co-sputtering with an indium-zinc target and a gallium target). The IGZO channel layer 808 may have a thickness of, for example, between about 30 nm and about 100 nm, such as about 50 nm.

In some embodiments, the IGZO channel layer 808 (and some of the other components shown in FIG. 8) may undergo an annealing process. In some embodiments, the annealing process includes a relatively low temperature (e.g., less than about 600° C., preferably less than about 450° C.) heating process in, for example, an ambient gaseous environment (e.g., nitrogen, oxygen, or ambient/air) to enhance the crystalline structure of the IGZO. The heating process may occur for between about 1 minute and about 200 minutes. After the annealing (or heating) process, the IGZO may (substantially) include crystalline IGZO (c-IGZO). In some embodiments, the c-IGZO is c-axis aligned crystal (CAAC) IGZO, as is commonly understood.

The source electrode (or region) 810 and a drain electrode 812 are formed above the IGZO channel layer 808. As shown, the source electrode 810 and the drain electrode 812 lie above, and partially overlap the ends of the IGZO channel layer 808. As will be appreciated by one skilled in the art, the source electrode 810 and the drain electrode 812 may be defined as shown in FIG. 8 using a “back-channel etch” (BCE) process to, for example, form the gap between the source electrode 810 and the drain electrode 812, which is vertically aligned with the gate electrode 804. However, in some embodiments, an etch-stop layer, as is commonly understood, may be formed above the IGZO channel layer 808 to facilitate the defining of the source electrode 810 and the drain electrode 812 (e.g., by protecting the IGZO during the etch process).

In some embodiments, the source electrode 810 and the drain electrode 812 are made of titanium, aluminum, molybdenum, copper, copper-manganese alloy, or a combination thereof. In some embodiments, the source electrode 810 and the drain electrode 812 include multiple sub-layers (e.g., sub-layers of titanium and titanium nitride). The source electrode 810 and the drain electrode 812 may have a thickness of, for example, between about 20 nm and 500 nm.

The passivation layer 814 is formed above the source electrode 810, the drain electrode 812, and the exposed portions of the gate dielectric layer 806 and the IGZO channel layer 808. In some embodiments, the passivation layer 814 is made of silicon oxide, silicon nitride, aluminum oxide, aluminum nitride, or a combination thereof and has a thickness of, for example, between about 0.1 micrometers (μm) and about 1.5 μm.

Although only a single device 800 is shown as being formed on a particular portion of the substrate 802 in FIG. 8, the manufacturing processes used to form the device 800 may be simultaneously performed on multiple portions of the substrate 100 such that multiple devices 800 are simultaneously formed, as is commonly understood. Further, although not shown, in some embodiments, such as those intended for use in display applications, the device 800 may also include pixel electrodes formed above the substrate 802. The pixel electrodes may be made of a transparent conductive material, such as indium-tin oxide (ITO).

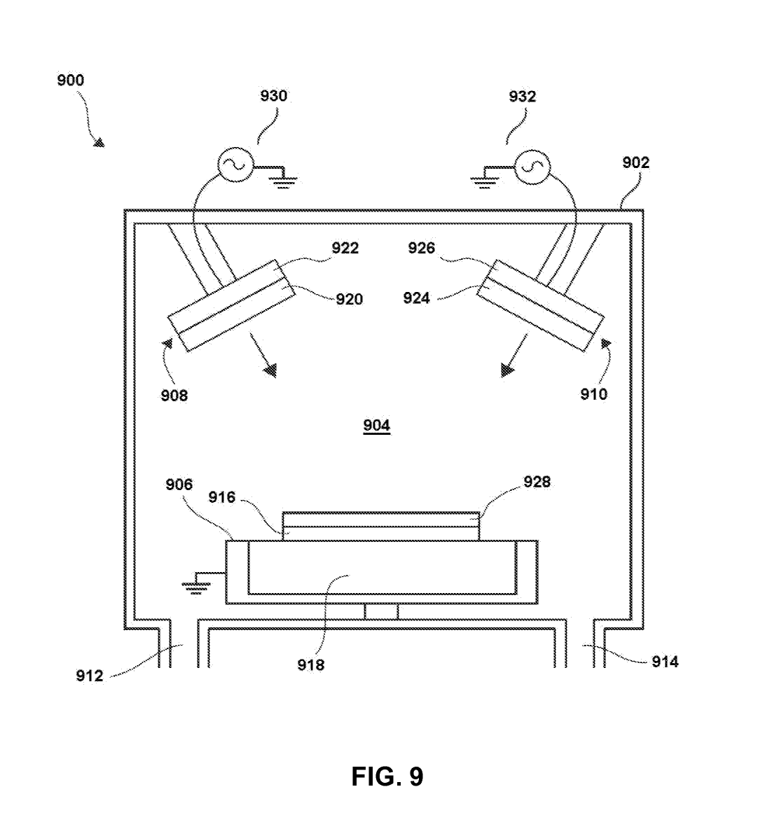

FIG. 9 provides a simplified illustration of a physical vapor deposition (PVD) tool (and/or system) 900 which may be used, in some embodiments, to form at least some components of the test devices (and/or IGZO devices) described above. The PVD tool 900 shown in FIG. 9 includes a housing 902 that defines, or encloses, a processing chamber 904, a substrate support 906, a first target assembly 908, and a second target assembly 910.

The housing 902 includes a gas inlet 912 and a gas outlet 914 near a lower region thereof on opposing sides of the substrate support 906. The substrate support 906 is positioned near the lower region of the housing 902 and in configured to support a substrate 916. The substrate 916 may be a round substrate having a diameter of, for example, about 200 mm or about 300 mm. In other embodiments (such as in a manufacturing environment), the substrate 916 may have other shapes, such as square or rectangular, and may be significantly larger (e.g., about 0.5 m to about 4 m across). The substrate support 906 includes a support electrode 918 and is held at ground potential during processing, as indicated.

The first and second target assemblies (or process heads) 908 and 910 are suspended from an upper region of the housing 902 within the processing chamber 904. The first target assembly 908 includes a first target 920 and a first target electrode 922, and the second target assembly 910 includes a second target 924 and a second target electrode 926. As shown, the first target 920 and the second target 924 are oriented or directed towards the substrate 916. As is commonly understood, the first target 920 and the second target 924 include one or more materials that are to be used to deposit a layer of material 928 on the upper surface of the substrate 916.

The materials used in the targets 920 and 924 may, for example, include indium, gallium, zinc, tin, silicon, platinum, silver, aluminum, magnesium, manganese, molybdenum, zirconium, hathium, titanium, copper, or any combination thereof (i.e., a single target may be made of an alloy of several metals). In some embodiments, the materials used in the targets may include oxygen, nitrogen, or a combination of oxygen and nitrogen in order to form oxides, nitrides, and oxynitrides. Additionally, although two targets 920 and 924 are shown, a different number of targets may be used (e.g., one or more than two targets).

The PVD tool 900 also includes a first power supply 930 coupled to the first target electrode 922 and a second power supply 932 coupled to the second target electrode 924. As is commonly understood, in some embodiments, the power supplies 930 and 932 pulse direct current (DC) power to the respective electrodes, causing material to be, at least in some embodiments, simultaneously sputtered (i.e., co-sputtered) from the first and second targets 920 and 924. In some embodiments, the power is alternating current (AC) to assist in directing the ejected material towards the substrate 916.

During sputtering, inert gases (or a plasma species), such as argon or krypton, may be introduced into the processing chamber 904 through the gas inlet 912, while a vacuum is applied to the gas outlet 914. The inert gas(es) may be used to impact the targets 920 and 924 and eject material therefrom, as is commonly understood. In embodiments in which reactive sputtering is used, reactive gases, such as oxygen and/or nitrogen, may also be introduced, which interact with particles ejected from the targets (i.e., to form oxides, nitrides, and/or oxynitrides).

Although not shown in FIG. 9, the PVD tool 900 may also include a control system having, for example, a processor and a memory, which is in operable communication with the other components shown in FIG. 9 and configured to control the operation thereof in order to perform the methods described herein.

Although the PVD tool 900 shown in FIG. 9 includes a stationary substrate support 906, it should be understood that in a manufacturing environment, the substrate 916 may be in motion (e.g., an in-line configuration) during the formation of various layers described herein.

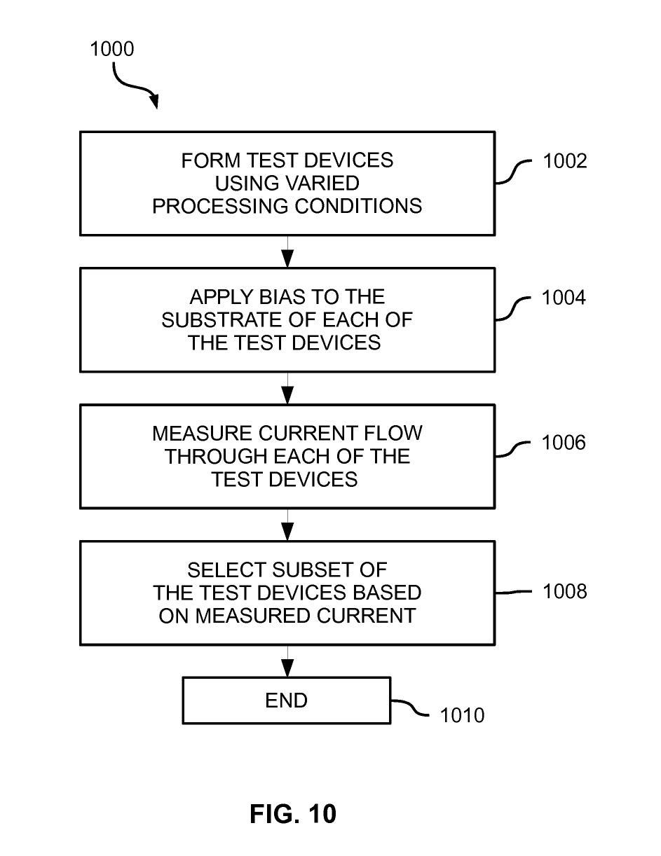

FIG. 10 illustrates evaluating IGZO with respect to NBIS according to some embodiments. At block 1002, the method 1100 begins with a plurality of test devices (or IGZO devices) being formed. The test devices may be similar to those described above. That is, in some embodiments, each of the test devices includes a semiconductor substrate (e.g., P++ doped silicon), a dielectric layer (e.g., silicon nitride) formed above the semiconductor substrate, a test layer (e.g., IGZO) formed above the dielectric layer, and at least one conductor (e.g., a transparent conductor) formed above the test layer.

In some embodiments, the test devices (or at least the IGZO layers therein) are formed using combinatorially varied processing conditions. That is, each of the test devices is formed using a respective one of a plurality of sets of processing conditions.

At block 1004, a bias is applied to the substrate of each of the test devices. The bias may be for example between about −10 V and about −20 V, and at block 1006, the current flow through each of the test devices is measured (while the bias is applied). At block 1008, a subset of the test devices (and/or the processing conditions used to form the test devices, or at least the IZGO therein) is selected based on the measured current(s) through the test devices.

As described above, the test devices exhibiting the lowest (or at least relatively low) current flow may be deemed to provide desirable performance with respect to NBIS, and the processing conditions used to form those test devices (or at least the IGZO therein) may be selected for further testing and/or use in IGZO devices, such as IGZO TFTs.

In some embodiments, within block 1004, the bias is applied to the substrate of each of the test devices while the test devices are illuminated by a light source (e.g., generating monochromatic light). Additionally, in some embodiments, the current flow through the test devices is (also) measured when the test devices are in a low light environment (with the bias applied) to establish a “baseline” current flow (e.g., without NBIS). Further, the method 1000 may also include additional steps/features, such as those described above with respect to FIGS. 1-7. At block 1010, the method ends.

Thus, in some embodiments, methods for measuring NBIS in IGZO are provided. A plurality of IGZO devices is formed. Each of the plurality of IGZO devices includes a semiconductor substrate and an IGZO layer formed above the semiconductor substrate. A processing condition used to form at least two of the plurality of IGZO devices is varied in a combinatorial manner. A bias is applied to the semiconductor substrate of each of the plurality of IGZO devices. A current flow through each of the plurality of IGZO devices while the bias is applied is measured.

In some embodiments, methods for measuring NBIS in IGZO are provided. A plurality of IGZO devices are provided. Each of the plurality of IGZO devices includes a semiconductor substrate and a test layer, including IGZO, formed above the semiconductor substrate. A processing condition used to form at least two of the plurality of IGZO devices is varied in a combinatorial manner. A bias is applied to the semiconductor substrate of each of the plurality of IGZO devices. A current flow through each of the plurality of IGZO devices while the bias is applied to the semiconductor substrate of each of the plurality of IGZO devices is measured. A subset of the plurality of IGZO devices is selected. The measured current flow through the IGZO devices of the selected subset of the plurality of IGZO devices is less than a threshold.

In some embodiments, methods for measuring NBIS in IGZO are provided. A plurality of IGZO devices are formed. The forming of each of the plurality of IGZO devices includes providing a semiconductor substrate and forming an IGZO layer above the semiconductor substrate. A processing condition associated with the forming of the IGZO layers of at least two of the plurality of IGZO devices is varied in a combinatorial manner. A bias is applied to the semiconductor substrate of each of the plurality of IGZO devices. A current flow through each of the plurality of IGZO devices while the bias is applied to the semiconductor substrate of each of the plurality of IGZO devices is measured. At least one processing condition associated with the forming of the IGZO layers of the plurality of IGZO devices is selected based on the measured current flow through each of the plurality of IGZO devices. The at least one selected processing condition is associated with those of the plurality of IGZO devices for which the measured current is less than a threshold.

Although the foregoing examples have been described in some detail for purposes of clarity of understanding, the invention is not limited to the details provided. There are many alternative ways of implementing the invention. The disclosed examples are illustrative and not restrictive.

What is claimed:

1. A method for evaluating indium-gallium-zinc oxide (IGZO) with respect to negative bias illumination stress (NBIS), the method comprising:
forming a plurality of IGZO devices, wherein each of the plurality of IGZO devices comprises a semiconductor substrate and an IGZO layer formed above the semiconductor substrate;
varying a processing condition used to form at least two of the plurality of IGZO devices in a combinatorial manner;
applying a bias to the semiconductor substrate of each of the plurality of IGZO devices; and
measuring a current flow through each of the plurality of IGZO devices while the bias is applied.

2. The method of claim 1, further comprising selecting a subset of the plurality of IGZO devices, wherein the measured current flow through the IGZO devices of the selected subset of the plurality of IGZO devices is less than a threshold.

3. The method of claim 1, wherein each of the plurality of IGZO devices further comprises:
a dielectric layer formed above the semiconductor substrate, wherein the IGZO layer is formed above the dielectric layer; and
at least one conductor formed above the IGZO layer.

4. The method of claim 1, wherein the varying of the processing condition used to form at least two of the plurality of IGZO devices comprises varying a processing condition used to form the IGZO layers of the plurality of IGZO devices.

5. The method of claim 4, further comprising selecting the processing conditions used to form the IGZO layers of the selected subset of the plurality of IGZO devices.

6. The method of claim 1, wherein the measuring of the current flow comprises measuring a current flow through the semiconductor substrate, the dielectric layer, the IGZO layer, and the at least one conductor of each of the plurality of IGZO devices.

7. The method of claim 6, wherein the measuring of the current flow comprises:
applying a first amount of illumination to each of the plurality of IGZO devices;
measuring a first current flow through each of the plurality of IGZO devices while the first amount of illumination is applied to the IGZO device;
applying a second amount of illumination to each of the plurality of IGZO devices, wherein the second amount of illumination is greater than the first amount of illumination; and
measuring a second current flow through each of the plurality of IGZO devices while the second amount of illumination is applied to the IGZO device.

8. The method of claim 7, further comprising comparing the second current flow to the first current flow for each of the plurality of IGZO devices.

9. The method of claim 3, wherein the semiconductor substrate of each of the plurality of IGZO devices comprises P++ doped silicon, and wherein the dielectric layer of each of the plurality of IGZO devices comprises silicon oxide, silicon nitride, or a combination thereof.

10. The method of claim 9, wherein the at least one conductor of each of the plurality of IGZO devices comprises platinum, graphene, zinc oxide, indium-tin oxide (ITO), or a combination thereof.

11. A method for evaluating indium-gallium-zinc oxide (IGZO) with respect to negative bias illumination stress (NBIS), the method comprising:
forming a plurality of IGZO devices, wherein each of the plurality of IGZO devices comprises a semiconductor substrate and a test layer comprising IGZO formed above the semiconductor substrate;
varying a processing condition used to form at least two of the plurality of IGZO devices in a combinatorial manner;
applying a bias to the semiconductor substrate of each of the plurality of IGZO devices;
measuring a current flow through each of the plurality of IGZO devices while the bias is applied to the semiconductor substrate of each of the plurality of IGZO devices; and
selecting a subset of the plurality of IGZO devices, wherein the measured current flow through the IGZO devices of the selected subset of the plurality of IGZO devices is less than a threshold.

12. The method of claim 11, wherein the semiconductor substrate of each of the plurality of IGZO devices comprises P++ doped silicon.

13. The method of claim 12, wherein the dielectric layer of each of the plurality of IGZO devices comprises silicon oxide, silicon nitride, or a combination thereof.

14. The method of claim 12, wherein the at least one conductor of each of the plurality of IGZO devices comprises platinum, graphene, zinc oxide, indium-tin oxide (ITO), or a combination thereof.

15. The method of claim 14, wherein the measuring of the current flow comprises:
applying a first amount of illumination to each of the plurality of IGZO devices;
measuring a first current flow through the semiconductor substrate, the dielectric layer, the test layer, and the at least one conductor of each of the plurality of IGZO devices while the first amount of illumination is applied to the IGZO device;
applying a second amount of illumination to each of the plurality of IGZO devices, wherein the second amount of illumination is greater than the first amount of illumination;
measuring a second current flow through the semiconductor substrate, the dielectric layer, the test layer, and the at least one conductor of each of the plurality of IGZO devices while the second amount of illumination is applied to the IGZO device; and
comparing the second current flow to the first current flow for each of the plurality of IGZO devices.

16. A method for evaluating indium-gallium-zinc oxide (IGZO) with respect to negative bias illumination stress (NBIS), the method comprising:
forming a plurality of IGZO devices, wherein the forming of each of the plurality of IGZO devices comprises:
providing a semiconductor substrate; and
forming an IGZO layer above the semiconductor substrate;
varying a processing condition associated with the forming of the IGZO layers of at least two of the plurality of IGZO devices in a combinatorial manner;
applying a bias to the semiconductor substrate of each of the plurality of IGZO devices;
measuring a current flow through each of the plurality of IGZO devices while the bias is applied to the semiconductor substrate of each of the plurality of IGZO devices; and
selecting at least one processing condition associated with the forming of the IGZO layers of the plurality of IGZO devices based on the measured current flow through each of the plurality of IGZO devices, wherein the at least one selected processing condition is associated with those of the plurality of IGZO devices for which the measured current is less than a threshold.

17. The method of claim 16, wherein the measuring of the current flow comprises:
applying a first amount of illumination to each of the plurality of IGZO devices;
measuring a first current flow through each of the plurality of IGZO devices while the first amount of illumination is applied to each of the plurality of IGZO devices;
applying a second amount of illumination to each of the plurality of IGZO devices, wherein the second amount of illumination is greater than the first amount of illumination; and
measuring a second current flow through each of the plurality of IGZO devices while the second amount of illumination is applied to each of the plurality of IGZO devices.

18. The method of claim 17, further comprising comparing the second current flow to the first current flow for each of the plurality of IGZO devices.

19. The method of claim 18, wherein the semiconductor substrate of each of the plurality of IGZO devices comprises P++ doped silicon.

20. The method of claim 19, wherein the forming of each of the plurality of IGZO devices further comprises:
forming a dielectric layer above the semiconductor substrate, wherein the dielectric layer comprises silicon oxide, silicon nitride, or a combination thereof and the IGZO layer is formed above the dielectric layer; and
forming at least one conductor above the IGZO layer, wherein the at least one conductor comprises platinum, graphene, zinc oxide, indium-tin oxide (ITO), or a combination thereof.
